(19) United States
(12) Patent Application Publication
(10) Pub. No.: US 2006/0051315 A1
(43) Pub. Date: Mar. 9, 2006

(54) Polymers for delivering peptides and small molecules in vivo

(76) Inventors: Puthupparampil V. Scaria, Montgomery Village, MD (US); Martin C. Woodle, Bethesda, MD (US)

Correspondence Address: HELLER EHRMAN WHITE & MCAULIFFE LLP, 1717 RHODE ISLAND AVE, NW, WASHINGTON, DC, 20036-3001, US

(21) Appl. No.: 10/502,986

(22) PCT Filed: Jan. 31, 2003
(86) PCT No.: PCT/US03/02710

§ 371 Date: Oct. 18, 2005

Related U.S. Application Data
(60) Provisional application No. 60/352,881, filed on Feb. 1, 2002.

(51) Int. Cl. A61K 38/17; C08G 63/91

(52) U.S. Cl. 424/78.17; 525/54.1; 525/54.2

(57) ABSTRACT

Certain hydrophilic polymers, such as a polyoxazoline, when conjugated to a polypeptide or small molecule agent, can enhance the bioavailability of the agent when administered in vivo. Accordingly, hydrophilic polymers of the invention can be used as a delivery vehicle to treat any number of disorders and/or confer a myriad of therapeutic benefits to a subject.

### BACKGROUND OF THE INVENTION

1. Field of the Invention

The invention relates to methods of delivering one or more polypeptide or small molecule therapeutic agents to a cell in conjunction with hydrophilic polymers or hydrophilic polymers and targeting ligands.

2. Background

Many barriers to effective polypeptide and small molecule administration to in vivo systems have been overcome. For instance, the use of vectors (e.g., viral vectors, polymers, nanoparticles, and liposomes) have shown great promise in counteracting the rapid rate of clearance of an administered agent from the blood.

Such delivery systems continue to face obstacles, however. For instance, immunogenicity and toxicity of viral vectors are among the barriers that limit their therapeutic application. To this end, a patient's immune defense often mounts a response to any administered viral vector particle, since the viral particles are produced via a natural packaging cell production. Such “natural packaging” produces particles virtually identical to those of the virus from which the vector is derived. The produced capsid or envelop, thus, is sensitive and susceptible to host immune defenses, which can affectively block the delivery of the recombinant genome.

Previous attempts to overcome these hurdles have involved tissue-specific targeting of a peptide via a non-invasive or minimally invasive route of administration. This approach aids in retaining therapeutic levels of an agent at the targeted tissue for prolonged time periods. Another focus is to allow protein clearance with minimal toxicity once the desired time period has elapsed.

Administered proteins and polypeptides often suffer from poor bio-availability, due to rapid removal of these molecules from blood circulation by enzymatic degradation. One technique for increasing efficacy of protein and other small molecule agents entails conjugating the administered agent with a polymer that can provide protection from enzymatic degradation in vivo, such as a polyethylene glycol (“PEG”) molecule. Such “PEGylation” often improves the circulation time and, hence, bio-availability of an administered agent.

PEG has shortcomings in certain respects, however. For example, because PEG is a linear polymer, the steric protection afforded by PEG is limited, as compared to branched polymers. Another major shortcoming of PEG is that it is only amenable to derivatization at its two terminals. This limits the number of other functional molecules (e.g., those helpful for protein or drug delivery to specific tissues) that can be conjugated to a PEG.

There is, accordingly, a need for a hydrophilic polymer that is capable of providing superior bioavailability of administered polypeptide- and small molecule agents. There also is a need for a hydrophilic polymer that is compatible with in vivo delivery systems, e.g., vectors, while maintaining the foregoing desired properties. The present invention satisfies these and other needs.

In addition there is a need to deliver therapeutic agents to specific tissues to achieve maximum therapeutic efficacy while minimizing toxic side effects. The present invention describes the delivery of therapeutic molecules to specific tissues by ligand-mediated delivery where the ligand and the therapeutic molecule are chemically conjugated to the hydrophilic polymers such as polyoxazoline, polyethylene glycol, polyacetal and others.

### SUMMARY OF THE INVENTION

Accordingly, it is an object of the invention to provide a hydrophilic polymer that increases bioavailability of a polypeptide or small molecule that is administered in vivo.

In another embodiment, the invention provides hydrophilic polymers which increase the bioavailibility of agents in an in vivo system, such as peptides, polypeptides, proteins and small molecules, drugs and nucleic acid drugs.

In another embodiment, the invention provides pharmaceutical compositions comprising one or more of the hydrophilic polymers described herein.

In another embodiment, the invention provides methods of increasing the bioavailability of a peptide or small molecule that is administered to an in vivo system, using a hydrophilic polymer.

In another preferred embodiment, the invention also provides methods for delivering a therapeutic agent to a subject in need thereof comprising administering to a subject in need thereof an effective amount of a compound according to any one of claims 1-4.

In another preferred embodiment, the instant polymer comprises a targeting ligand or moiety for targeting specific cells and tissues.

In another preferred embodiment, the instant polymer comprises a fusogenic ligand or moiety for facilitating entry of an agent, preferably a nucleic acid, into a nucleus of a cell.

In another preferred embodiment, the instant polymer comprises a nuclear targeting ligand or moiety for targeting specific cells and tissues.

These and other objects will become apparent to a skilled worker by reference to the specification and conventional teachings in the art.

### DETAILED DESCRIPTION OF THE PREFERRED EMBODIMENTS

The present inventors surprisingly have discovered that in vivo bioavailability of a therapeutic agent can be increased if conjugated to one of a group of hydrophilic polymers. In this sense, the polymer, which preferably is a polyoxazoline, acts as a delivery vehicle for a therapeutic agent. A therapeutic agent is a nucleic acid fragment, peptide, polypeptide, protein or other small molecule drug. By “Peptide” or “polypeptide” is meant two or more amino acids linked to each other via a peptide bond. As used herein, a “small molecule” or “small molecule drug” or “small molecule agent”, or “therapeutic drug” or a “drug” means an organic molecule, other than a nucleic acid molecule that, when administered to a mammal (e.g., human being), confers a therapeutic benefit. A polymer for use in the invention preferably is conjugated to (i) a nucleic acid, polypeptide or small molecule drug; and (ii) one or more other moieties, e.g., a ligand or tissue-targeting domain, yet retains (or substantially retains) its desired characteristics. These features, therefore, render the polymers disclosed herein, e.g., polyoxazolines, polyethylene glycol suitable for multiple routes of administration, ranging from oral, to systemic, to local administrations.

As used herein (unless specified to the contrary), the term “polymer” or “polymer of the invention” preferably means a hydrophilic polymer represented by any of the following structures.

In a preferred embodiment, a polymer of the invention may be represented by the following:
R¹—X—R²
wherein,
X may be a hydrophilic polymer such as polyoxazoline, polyethylene glycol, polyacetal, polylactic acid, polyglycolic acid;
R¹ may be a hydroxyl group, a sulfhydryl group, carboxylic acid, carboxylic acid ester, amino, amide group, a cell targeting ligand, or a tissue targeting ligand; and
R² may be a therapeutic agent selected from the group consisting of a peptide, polypeptide, protein, nucleic acid or a therapeutic drug,

In another preferred embodiment, a polymer of the invention may be represented by the following:
R²—X—R¹
wherein,
X may be a hydrophilic polymer such as polyoxazoline, polyethylene glycol, polyacetal, polylactic acid, polyglycolic acid,
R¹ may be a hydroxyl group, a sulfhydryl group, carboxylic acid, carboxylic acid ester, amino, amide group, a cell targeting ligand, or a tissue targeting ligand; and
R² may be a therapeutic agent selected from the group consisting of a peptide, polypeptide, protein, nucleic acid or a therapeutic drug.

In another preferred embodiment, a polymer of the invention may be represented by the following: 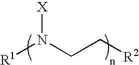
wherein,
X may be —CO—R, —(CH₂)ₘ—COOH, wherein m is an integer 1-25, (CH₂)ₚ—OH, wherein p is an integer 1-25, —(CH₂)_{q}—COOH, wherein q is an integer 1-25, an ester group, such as carboxylic acid esters, polyethylene glycol, polylactic acid, polyglycolic acid, polyoxazoline, amino, imidazole, or guanidinium; wherein R may be —CH₃, —C₂H₅, —(CH₂)ᵣ—OH, wherein r is an integer 1-25;
R may be a hydroxyl group, a sulfhydryl group, carboxylic acid, carboxylic acid ester, amino, amide group, a targeting moiety, a fusogenic moiety, or a nuclear targeting moiety; and
R² may be a therapeutic agent selected from the group consisting of a peptide, polypeptide, protein, nucleic acid or a therapeutic drug, and
n is 1-500.

In another preferred embodiment, a polymer of the instant invention may also be represented by the following: 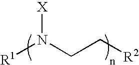
wherein,
X may be —CO—R, —(CH₂)ₘ—COOH, wherein m is an integer 1-25, (CH₂)ₚ—OH, wherein p is an integer 1-25, —(CH₂)_{q}—COOH, wherein q is an integer 1-25, an ester group, such as carboxylic acid esters, polyethylene glycol, polylactic acid, polyglycolic acid, polyoxazoline, amino, imidazole, or guanidinium; wherein R may be —CH₃, —C₂H₅, or —(CH₂)ₙ—OH, wherein r is an integer 1-25;
R¹ may be a therapeutic agent selected from the group consisting of a peptide, polypeptide, protein, nucleic acid or a therapeutic drug; and
R² may be a hydroxyl group, a sulfhydryl group, carboxylic acid, carboxylic acid ester, amino, an amide group, a targeting moiety, a fusogenic moiety, or a nuclear targeting moiety; and
n=1-500.

In another preferred embodiment, a polymer of the instant invention may also be represented by the following: 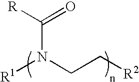
wherein,
R may be —CH₃, —C₂H₅, a hydrocarbon with 1-18 carbons, (C₁-C₂₅)—OH, (C₁-C₂₅)—COOH, polyethyleneglycol, polylactic acid, polyglycolic acid, polyoxazoline;
R¹ may be a hydroxyl group, a sulfhydryl group, carboxylic acid, carboxylic acid ester, amino, amide group, a cell targeting ligand, or a tissue targeting ligand; and
R² may be a therapeutic agent selected from the group consisting of a peptide, polypeptide, protein, nucleic acid or a therapeutic drug, and
n=1-500.

In another preferred embodiment, a polymer of the instant invention may also be represented by the following: 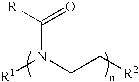
wherein,
R may be —CH₃, —C₂H₅, a hydrocarbon with 1-18 carbons, (C₁-C₂₅)—OH, (C₁-C₂₅)—COOH, polyethyleneglycol, polylactic acid, polyglycolic acid, polyoxazoline;
R¹ may be a therapeutic agent selected from the group consisting of a peptide, polypeptide, protein, nucleic acid or a therapeutic drug;
R² may be a hydroxyl group, a sulfhydryl group, carboxylic acid, carboxylic acid ester, amino, amide group, a targeting moiety, a fusogenic moiety or a nuclear targeting moiety, and
n=1-500.

In a preferred embodiment, the polymer may be a polyoxazoline, which is a species embraced by the foregoing polymers. A polyoxazoline may be represented by the following: 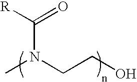
wherein,
R may be —CH₃ for polymethyloxazoline (PMOZ), or
R may be —CH₂CH₃ for polyethyloxazoline (PEOZ), and
n is 1-500.

In the above structure, R¹ is preferably bound to the left side of the molecule.

In another preferred embodiment, a polymer of the instant invention may also be represented by the following 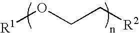
wherein,
R¹ may be a therapeutic agent selected from the group consisting of a peptide, polypeptide, protein, nucleic acid or a therapeutic drug;
R² may be a hydroxyl group, a sulfhydryl group, carboxylic acid, carboxylic acid ester, amino, amide group, a targeting moiety, a fusogenic moiety or a nuclear targeting moiety, and
n=1-500.

In another preferred embodiment, a polymer of the instant invention may also be represented by the following: 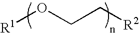
wherein,
R¹ may be a hydroxyl group, a sulfhydryl group, carboxylic acid, carboxylic acid ester, amino, amide group, a cell targeting ligand, or a tissue targeting ligand; and
R² may be a therapeutic agent selected from the group consisting of a peptide, polypeptide, protein, nucleic acid or a therapeutic drug, and
n=1-500.

Nucleic acid refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral methyl phosphonates, 2-O-methyl ribonucleotides, and peptide-nucleic acids (PNAs).

A polyoxazoline or hydrophilic polymer of the invention also is capable of having multiple ligands conjugated onto the distal ends of the polymer. This can, for instance, enhance selective tissue and cellular interactions, thereby minimizing the interaction of an administered agent and non-targeted tissues and cells.

A targeting ligand enhances binding of the polymer to target tissue or cells and permits highly specific interaction of the polymers with the target tissue or cell. In one embodiment, the polymer will include a ligand effective for ligand-specific binding to a receptor molecule on a target tissue and cell surface (Woodle et al., Small molecule ligands for targeting long circulating liposomes, in Long Circulating Liposomes: Old drugs, new Therapeutics, Woodle and Storm eds., Springer, 1998, p 287-295).

The polymer may include two or more targeting moieties, depending on the cell type that is to be targeted. Use of multiple targeting moieties can provide additional selectivity in cell targeting, and also can contribute to higher affinity and/or avidity of binding of the polymer to the target cell. When more than one targeting moiety is present on the polymer, the relative molar ratio of the targeting moieties may be varied to provide optimal targeting efficiency. Methods for optimizing cell binding and selectivity in this fashion are known in the art. The skilled artisan also will recognize that assays for measuring cell selectivity and affinity and efficiency of binding are known in the art and can be used to optimize the nature and quantity of the targeting ligand(s).

Suitable ligands include, but are not limited to: vascular endothelial cell growth factor for targeting endothelial cells: FGF2 for targeting vascular lesions and tumors; somatostatin peptides for targeting tumors; transferrin for targeting umors; melanotropin (alpha MSH) peptides for tumor targeting; ApoE and peptides for LDL receptor targeting; von Willebrand's Factor and peptides for targeting exposed collagen; Adenoviral fiber protein and peptides for targeting Coxsackie-adenoviral receptor (CAR) expressing cells; PD1 and peptides for targeting Neuropilin 1; EGF and peptides for targeting EGF receptor expressing cells; and RGD containing peptides and their analogues for targeting integrin expressing cells.

Other examples include (i) folate, where the polymer is intended for treating tumor cells having cell-surface folate receptors, (ii) pyridoxyl, where the polymer is intended for treating virus-infected CD4+ lymphocytes, or (iii) sialyl-Lewis, where the polymer is intended for treating a region of inflammation. Other peptide ligands may be identified using methods such as phage display (F. Bartoli et al., Isolation of peptide ligands for tissue-specific cell surface receptors, in Vector Targeting Strategies for Therapeutic Gene Delivery (Abstracts form Cold Spring Harbor Laboratory 1999 meeting), 1999, p4) and microbial display (Georgiou et al., Ultra High Affinity Antibodies from Libraries Displayed on the Surface of Microorganisms and Screened by FACS, in Vector Targeting Strategies for Therapeutic Gene Delivery (Abstracts form Cold Spring Harbor Laboratory 1999 meeting), 1999, p 3.).

In an exemplary embodiment, the targeting ligand may be somatostatin or a somatostatin analog. Somatostatin has the sequence AGCLNFFWKTFTSC, and contains a disulfide bridge between the cysteine residues. Many somatostatin analogs that bind to the somatostatin receptor are known in the art and are suitable for use in the present invention, such as those described, for example, in U.S. Pat. No. 5,776,894, which is incorporated herein by reference in its entirety. Particular somatostatin analogs that are useful in the present invention are analogs having the general structure F*CY-(DW)KTCT, where DW is D-tryptophan and F* indicates, that the phenylalanine residue may have either the D- or L-absolute configuration. As in somatostatin itself, these compounds are cyclic due to a disulfide bond between the cysteine residues. Advantageously, these analogs may be derivatized at the free amino group of the phenylalanine residue, for example with a polycationic moiety such as a chain of lysine residues. The skilled artisan will recognize that other somatostatin analogs that are known in the art may advantageously be used in the invention.

Furthermore, methods have been developed to create novel peptide sequences that elicit strong and selective binding for target tissues and cells such as “DNA Shuffling” (W. P. C. Stremmer, Directed Evolution of Enzymes and Pathways by DNA Shuffling, in Vector Targeting Strategies for Therapeutic Gene Delivery (Abstracts form Cold Spring Harbor Laboratory 1999 meeting), 1999, p. 5.) and these novel sequence peptides are suitable ligands for the invention. Other chemical forms for ligands are suitable for the invention such as natural carbohydrates which exist in numerous forms and are a commonly-used ligand by cells (Kraling et al., Am. J. Path. 150:1307 (1997) as well as novel chemical species, some of which may be analogues of natural ligands such as D-amino acids and peptidomimetics and others which are identifed through medicinal chemistry techniques such as combinatorial chemistry (P. D. Kassner et al., Ligand Identification via Expression (LIVE): Direct selection of Targeting Ligands from Combinatorial Libraries, in Vector Targeting Strategies for Therapeutic Gene Delivery (Abstracts form Cold Spring Harbor Laboratory 1999 meeting), 1999, p8.).

The targeting moiety provides tissue- and cell-specific binding. The ligands may be covalently attached to the polymer so that exposure is adequate for tissue and cell binding. For example, a peptide ligand can be covalently coupled to a polymer such as polyoxazoline or polyethylene glycol or other hydrophilic polymers.

The number of targeting molecules present on the outer layer will vary, depending on factors such as the avidity of the ligand-receptor interaction, the relative abundance of the receptor on the target tissue and cell surface, and the relative abundance of the target tissue and cell. Nevertheless, a targeting molecule coupled with each polymer usually provides suitable enhancement of cell targeting.

The presence of the targeting moiety leads to the desired enhancement of binding to target tissue and cells. An appropriate assay for such binding may be ELISA plate assays, cell culture expression assays, or any other binding assays known in the art.

The fusogenic moiety promotes fusion of the polymer to the cell membrane of the target cell, facilitating entry of the polymer and therapeutic agents into the cell. In one embodiment, the fusogenic moiety comprises a fusion-promoting element. Such elements interact with cell membranes or endosome membranes in a manner that allows transmembrane movement of large molecules or particles, or disrupts the membranes such that the aqueous phases that are separated by the membranes may freely mix. Examples of suitable fusogenic moieties include, but are not limited to membrane surfactant peptides, e.g. viral fusion proteins such as hemagglutinin (HA) of influenza virus, or peptides derived from toxins such as PE and ricin. Other examples include sequences that permit cellular trafficking such as HIV TAT protein and antennapedia or those derived from numerous other species, or synthetic polymers that exhibit pH sensitive properties such as poly(ethylacrylic acid) (Lackey et al., Proc. Int. Symp. Control. Rel. Bioact. Mater. 1999, 26, #6245), N-isopropylacrylamide methacrylic acid copolymers (Meyer et al., FEBS Lett. 421:61 (1999)), or poly(amidoamine)s, (Richardson et al., Proc. Int. Symp. Control. Rel. Bioact. Mater. 1999, 26, #251), and lipidic agents that are released into the aqueous phase upon binding to the target cell or endosome. Suitable membrane surfactant peptides include an influenza hemagglutinin or a viral fusogenic peptide such as the Moloney murine leukemia virus (“MoMuLV” or MLV) envelope (env) protein or vesicular stroma virus (VSV) G-protein. The membrane-proximal cytoplasmic domain of the MoMuLV env protein may be used. This domain is conserved among a variety of viruses and contains a membrane-induced α-helix.

Suitable viral fusogenic peptides for the instant invention may include a fusion peptide from a viral envelope protein ectodoinain, a membrane-destabilizing peptide of a viral envelope protein membrane-proximal domain, hydrophobic domain peptide segments of so called viral “fusion” proteins, and an amphiphilic-region containing peptide. Suitable amphiphilic-region containing peptides include, but are not limited to: melittin, the magainins, fusion segments from H. influenza hemagglutinin (HA) protein, HIV segment I from the cytoplasmic tail of HIV1 gp41, and amphiphilic segments from viral env membrane proteins including those from avian leukosis virus (ALV), bovine leukemia virus (BLV), equine infectious anemia (EIA), feline immunodeficiency virus (FIV), hepatitis virus, herpes simplex virus (HSV) glycoprotein H; human respiratory syncytia virus (hRSV), Mason-Pfizer monkey virus (MPMV), Rous sarcoma virus (RSV), parainfluenza virus (PINF), spleen necrosis virus (SNV), and vesicular stomatitis virus (VSV). Other suitable peptides include microbial and reptilian cytotoxic peptides. The specific peptides or other molecules having greatest utility can be identified using four kinds of assays: 1) ability to disrupt and induce leakage of aqueous markers from liposomes composed of cell membrane lipids or fragments of cell membranes, 2) ability to induce fusion of liposomes composed of cell membrane lipids or fragments of cell membranes, 3) ability to induce cytoplasmic release of particles added to cells in tissue culture, and 4) ability to enhance plasmid expression by particles in vivo tissues when administered locally or systemically.

The fusogenic moiety also may be comprised of a polymer, including peptides and synthetic polymers. In one embodiment, the peptide polymer comprises synthetic peptides containing amphipathic aminoacid sequences such as the “GALA” and “KALA” peptides (Wyman T B, Nicol F, Zelphati O, Scoria P V, Plank C, Szoka F C Jr, Biochemistry 1997, 36:3008-3017; Subbarao N K, Parente R A, Szoka F C Jr, Nadasdi L, Pongracz K, Biochemistry 1987 26:2964-2972 or Wyman supra, Subbarao supra). Other peptides include non-natural aminoacids, including D aminoacids and chemical analogues such as peptoids. Suitable polymers include molecules containing amino or imidazole moieties with intermittent carboxylic acid functionalities such as ones that form “salt-bridges,” either internally or externally, including forms where the bridging is pH sensitive. Other polymers can be used including ones having disulfide bridges either internally or between polymers such that the disulfide bridges block fusogenicity and then bridges are cleaved within the tissue or intracellular compartment so that the fusogenic properties are expressed at those desired sites. For example, a polymer that forms weak electrostatic interactions with a positively charged fusogenic polymer that neutralizes the positive charge could be held in place with disulfide bridges between the two molecules and these disulfides cleaved within an endosome so that the two molecules dissociate releasing the positive charge and fusogenic activity. Another form of this type of fusogenic agent has the two properties localized onto different segments of the same molecule and thus the bridge is intramolecular so that its dissociation results in a structural change in the molecule. Yet another form of this type of fusogenic agent has a pH sensitive bridge.

The fusogenic moiety also may comprise a membrane surfactant polymer-lipid conjugate. Suitable conjugates include Thesit™, Brij 58™, Brij 78™, Tween 80™, Tween 20™, C₁₂E₈, C₁₄E₈, C₁₆E₈ (CₙEₙ,=hydrocarbon poly(ethylene glycol)ether where C represents hydrocarbon of carbon length N and E represents poly(ethylene glycol) of degree of polymerization N), Chol-PEG 900, analogues containing polyoxazoline or other hydrophilic polymers substituted for the PEG, and analogues having fluorocarbons substituted for the hydrocarbon. Advantageously, the polymer will be either biodegradable or of sufficiently small molecular weight that it can be excreted without metabolism. The skilled artisan will recognize that other fusogenic moieties also may be used without departing from the spirit of the invention.

Certain therapeutic agents exert their biological activity in the cell nucleus. Advantageously, when the intended biological target of a nucleic acid is the nucleus, the nucleic targeting moiety of the invention is “nuclear targeted,” that is, it contains one or more molecules that facilitate entry of the nucleic acid through the nuclear membrane into the nucleus of the host cell. Such nuclear targeting may be achieved by incorporating a nuclear membrane transport peptide, or nuclear localization signal (“NLS”) peptide, or small molecule that provides the same NLS function, into the core complex. Suitable peptides are described in, for example, U.S. Pat. Nos. 5,795,587 and 5,670,347 and in patent application WO 9858955, which are hereby incorporated by reference in their entirety, and in Aronsohn et al., J. Drug Targeting 1:163 (1997); Zanta et al., Proc. Nat'l Acad. Sci. USA 96:91-96 (1999); Ciolina et al., Targeting of Plasmid DNA to Importin alpha by Chemical coupling with Nuclear Localization SigIal Peptides, in Vector Targeting Strategies for Therapeutic Gene Delivery (Abstracts from Cold Spring Harbor Laboratory 1999 meeting), 1999, p 20; Saphire et al., J. Biol Chem; 273:29764 (1999). A nuclear targeting peptide may be a nuclear localization signal peptide or nuclear membrane transport peptide and it may be comprised of natural aminoacids or non-natural ammoacids including D aminoacids and chemical analogues such as peptoids. The NLS may be comprised of aminoacids or their analogues in a natural sequence or in reverse sequence. Another embodiment provides a steroid receptor-binding NLS moiety that activates nuclear transport of the receptor from the cytoplasm, wherein this transport carries the nucleic acid with the receptor into the nucleus.

In another embodiment, the NLS is coupled to the polymer in such a manner that the polymer is directed to the cell nucleus where it permits entry of a nucleic acid into the nucleus.

In another embodiment, incorporation of the NLS moiety into the polymer occurs through association with the nucleic acid, and this association is retained within the cytoplasm. This minimizes loss of the NLS function due to dissociation with the nucleic acid and ensures that a high level of the nucleic acid is delivered to the nucleus. Furthermore, the association with the nucleic acid does not inhibit the intended biological activity within the nucleus once the nucleic acid is delivered.

In yet another embodiment, the intended target of the biological activity of the nucleic acid is the cytoplasm or an organelle in the cytoplasm such as ribosomes, the golgi apparatus, or the endoplasmic reticulum. In this embodiment, a localization signal is included in the polymer or anchored to it so that it provides direction of the nucleic acid to the intended site where the nucleic acid exerts its activity. Signal peptides that can achieve such targeting are known in the art.

By virtue of its structure and chemical properties, a polymer of the invention, for example polyoxazoline, can provide advantages over conventionally used hydrophilic polymers, such as PEG. Preferred polyoxazolines of the inventions are polymethyloxazoline and polyethyloxazoline.

A polymer of the invention can be constructed and used as a linear polymer. Such polymers provide several advantages over commonly used hydrophilic polymer, such a PEG. For instance, polyoxazoline, when attached to a therapeutic agent, can provide longer blood circulation time for the agent. A polyoxazoline or other polymer of the invention also can provide hydrophilicity to a hydrophobic drug, which enhances bioavailability of the drug. The nitrogens in the back bone are amenable to substitutions. A PEG polymer backbone, on the other hand, contains oxygen atoms that are as amenable to substitutions. Accordingly, the hydrophobicity of a polyoxazoline can be modulated according to conventional means. Atoms such as nitrogens in the backbone also can be used to attach other functional molecules such as ligands, which can target the therapeutic agent or drug to specific tissues and cells.

Nitrogens in the backbone also can be used to introduce branching of the polymer. It is expected that the network of branching will protect an administered agent to a greater extent in vivo than other polymers (e.g., PEG), which will result in enhanced bioavailability. The branched structure of a polyoxazoline or other polymer of the invention, thus, can provide a desired effect on pharmacology, such as: increased circulation in the blood, increased affinity for cellular binding and uptake, and/or facilitated adsorption into the tissue and cell.

As indicated, functional moieties can be added to a polymer, e.g., polyoxazoline, at the nitrogens in the polymer backbone, the latter being amenable to derivatization. Functional moieties suitable for attachment to a polyoxazoline (either alone or in comibination) include: vascular endothelial growth factors, somatostatin and somatostatin analogs, transferring, melanotropin, ApoE and ApoE peptides, von Willebrand's factor and von Willebrand's factor peptides, adeno viral fiber protein and adenoviral fiber protein peptides, PD1 and PD1 peptides, EGF and EGF peptides, RGD peptides, CCK peptides, antibody and antibody fragments, folate, pyridoxyl and sialyl-Lewis X and chemical analogs thereof. A polypeptide or other small molecule preferably is attached at the terminal ends of the polymer.

Polymers of the present invention can possess one or more asymmetric carbon atoms and are thus capable of existing in the form of optical isomers as well as in the form of racemic or nonracemic mixtures thereof. The optical isomers can be obtained by resolution of the racemic mixtures according to conventional processes, for example by formation of diastereoisomeric salts by treatment with an optically active acid or base. Examples of appropriate acids are tartaric, diacetyltartaric, dibenzoyltartaric, ditoluoyltartaric and camphorsulfonic acid and then separation of the mixture of diastereoisomers by crystallization followed by liberation of the optically active bases from these salts. A different process for separation of optical isomers involves the use of a chiral chromatography column optimally chosen to maximize the separation of the enantiomers. Still another available method involves synthesis of covalent diastereoisomeric molecules by reacting the instant polymers with an optically pure acid in an activated form or an optically pure isocyanate. The synthesized diastereoisomers can be separated by conventional means such as chromatography, distillation, crystallization or sublimation, and then hydrolyzed to deliver the enantiomerically pure compound. The optically active compounds can likewise be obtained by utilizing optically active starting materials. These isomers may be in the form of a free acid, a free base, an ester or a salt.

The compounds of the present invention can be used in the form of salts derived from inorganic or organic acids. These salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, tosylate, mesylate and undecanoate. Also, the basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and other. Water or oil-soluble or dispersible products are thereby obtained.

Examples of acids which may be employed to form pharmaceutically acceptable acid addition salts include such inorganic acids as hydrochloric acid, sulphuric acid and phosphoric acid and such organic acids as oxalic acid, maleic acid, succinic acid and citric acid. Other examples include salts with alkali metals or alkaline earth metals, such as sodium, potassium, calcium or magnesium or with organic bases.

Methods of Making Polyoxazoline-Agent Conjugates.

A polymer of the invention, such as polyoxazoline, is conjugated, e.g., chemically, to a polypeptide or small molecule agent prior to in vivo administration. With reference to the following structures, the following protocol is a non-limiting method of preparing a polyoxazoline-agent conjugate of the invention.

Polymerization of the 2-oxazoline can be carried out using ICH₂CO₂Et as the initiator. The monomer (0.1 mmol) is taken in a dry glass tube and an equal volume of dry acetonitrile is added thereto. Depending on the degree of polymerization desired, a suitable amount of initiator is added to the above solution (e.g., 0.001 for a degree of polymerization of 100). The mixture is sealed under nitrogen at 80° for about 24 hours. Since oxazoline polymerization does not terminate without a chain terminator, a methanolic solution of KOH (0.5M) is added as chain terminator.

The resulting polyoxazoline with a carboxylic acid end can be conjugated to a free amino group or a hydroxyl group of a peptide, protein or drug as follows. To a 1 molar equivalent of the polymer (based on the carboxylic acid residue) is added 1.1 molar equivalents of dicyclohexyl carbodiimide(DCCI) and N-hydroxysuccinimide (NHS) at 0° C. To this, 1 molar equivalent of peptide or protein is added. The mixture is stirred at 0° C. for about one hour and then overnight at room temperature. The white precipitate of DCHU, is filtered off and the filtrate is evaporated to dryness. The conjugate is redissolved in water and dialyzed to remove small molecule impurities.

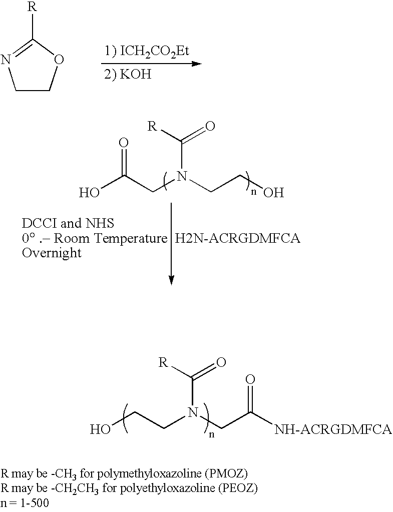

A polyoxazoline also can be conjugated to one or more moieties, such as tissue targeting molecules, including: vascular endothelial growth factors, somatostatin and somatostatin analogs, transferrin, melanotropin, ApoE and ApoE peptides, von Willebrand's factor and von Willebrand's factor peptides, adeno viral fiber protein and adenoviral fiber protein peptides, PD1 and PD1 peptides, EGF and EGF peptides, RGD peptides, CCK peptides, antibody and antibody fragments, folate, pyridoxyl and sialyl-Lewis X and chemical analogs thereof. Polyoxazoline can also be conjugated to endosome disrupting molecules such as fusogenic moiety of a viral peptide selected from the group consisting of MLV envelope protein, HA env peptide, a viral envelope protein ectodomain, a membrane—destabilizing domain of viral envelope protein, and hydrophobic domain of a viral fusion protein.

These molecules can be attached to the end of the polymer that is opposite to the end where the therapeutic molecule is attached. The order of attachment and synthetic strategies, which will be apparent to skilled worker in, the field, is determined based on the chemical properties of the molecules to be attached. To attach peptides and proteins, synthetic schemes similar to those shown in the above described protocol may be employed. For other molecules, the skilled artisan will recognize that other synthetic strategies will be used depending on the functional groups available for conjugation on these molecules.

Therapeutic Methods.

A polymer-agent conjugate, such as an agent-polyoxazoline conjugate (optionally attached to other moieties), can be used in a variety ways to bring about a therapeutic effect. A polyoxazoline-agent conjugate is particularly suitable for delivering an effective amount of a therapeutic agent to an in vivo system over an extended period of time. This finding is significant, given the limitations of state of the art delivery compositions. As a result, the drug and gene delivery vehicles of the invention can be useful in a number of therapeutic applications, including: therapeutic vaccines, preventative vaccines, treatment of inflammatory disorders and many types of malignancies, as well as any other regimen involving repeated administration of a therapeutic agent, which is any agent which elicits a beneficial response or alleviates symptoms of a disease or disorder and includes peptides, polypeptides, proteins, nucleic acids and small molecule drugs.

The present invention provides methods of administering one or more therapeutic small molecules or polypeptides to a subject, using a vehicle comprised of a polyoxazoline, to bring about a therapeutic benefit to the subject. As used herein, a “therapeutic small molecule” or “therapeutic polypeptide” is any small molecule or polypeptide that can confer a therapeutic benefit to a subject. In the present invention, a therapeutic small molecule or polypeptide also can be administered to a subject in conjunction with a synthetic vector. The subject preferably is mammalian such as a mouse, and more preferably is a human being.

Delivery vehicles for use in the present invention can be used to stimulate an immune response, which may be protective or therapeutic. Accordingly, the delivery vehicles can be used to vaccinate a subject against an antigen.

In this sense, the invention provides methods for vaccinating or enhancing a physiological response against a pathogen in a subject. This methodology can entail administering to the subject a first, or priming, dosage of a therapeutic peptide, followed by administering to the subject one or more booster dosages of the therapeutic peptide.

The administration regimen can vary, depending on, for example, (i) the subject to whom the therapeutic agent is administered, and (ii) the pathogen that is involved. For instance, a booster dosage of a therapeutic peptide may administered about two weeks after priming, followed by successive booster dosages, which can occur between intervals of constant or increasing duration. It is desirable to administer therapeutic peptide molecules at a periodicity that is appropriate according to the subject's immune response.

In the preceding administration steps, the administered peptide molecule is conjugated to a polymer of the invention. Preferably, the therapeutic peptide molecule in the foregoing steps elicits a humoral and/or cellular response in the subject, causing the subject to exhibit a degree of immunity against the pathogen that is greater than before the therapeutic method is carried out.

The antigen against which the subject exhibits an increased immunity can be the peptide antigen that is administered. Alternatively, the antigen against which the subject exhibits an increased immunity is distinct from, or in addition to, the administered peptide antigen. In the latter approach, for instance, the peptide antigen can act to enhance an immune response against another antigen, e.g., a component of a tumor.

The route of administration may vary, depending on the therapeutic application (e.g., preventative or therapeutic vaccine) and the type of disorder to be treated. The peptide delivery vehicle may be injected into the skin, muscle, intravenously, directly to the portal, hepatic vein or bile duct, locally to a tumor or to a joint, or orally.

An administered therapeutic peptide molecule also may induce an immune response. A response can be achieved to intracellular infectious agents including, for example, tuberculosis, Lyme disease, and others. A response can be achieved by delivery of an antigen, cytokines, or a combination thereof. The invention also provides for the delivery of HIV antigens and induction of both a protective and a therapeutic immune response for preventing and treating HIV, respectively.

The invention additionally provides for the delivery of antigens that elicit a humoral and/or a cellular immune response. This heightened immune response can provide protection from a challenge with infectious agents characterized as containing or displaying the antigen. In one embodiment, the therapeutic agent is a cytokine, which may or may not be co-administered with another antigen. A cytokine acts to recruit an immune response, which can enhance an immune response to an expressed antigen. Accordingly, cytokine administration according to the invention can induce APCs and other immune response cells to the vicinity of tumor cells, in which case there is no requirement for co-adminstration of an antigen. Yet, in another embodiment, one or more antigens and cytokines can be co-administered.

Accordingly, the invention contemplates the use of immunostimulatory cytokines, as well as protein analogues exhibiting biological activity similar to an immunostimulatory cytokine, to vaccinate a subject. Suitable cytokines for use in enhancing an immune response include GM-CSF, IL-1, IL-12, IL-15, IL-2, interferons, B-40, B-7, tumor necrosis factor (TNF) and others. The invention also contemplates utilizing therapeutic agents that can down-regulate immunosupressant cytokines. The invention also provides for administration of “recruitment cytokines” at tumors, which can initiate a cellular immune response at the tumor site, giving recognition and killing of tumor cells at the site of expression and at distal tumor sites.

A polyoxazoline also may be used to deliver an agent that treats a disorder characterized by inflammation. In one approach, one or more therapeutic agents is administered to a subject suffering from a disorder characterized by inflammation, in order to suppress or retard an immune response. Treatable disorders include rheumatoid arthritis, psoriasis, gout and inflammatory bowel disorders. Suitable therapeutic agents for use in treating inflammation include inflammation inhibitory cytokines, such as: IL-IRA, soluble TNF receptor, and soluble Fas ligand.

The route and site of administration will vary, depending on the disorder and the location of inflammation. The polyoxazoline-agent, with or without a synthetic vector, can be administered into a joint; administration thereto can be in conjunction with electroporation.

Pharmaceutical Compositions

The amount of active ingredient that may be combined with the carrier materials to produce a single dosage form will vary depending upon the host treated and the particular mode of administration.

The dosage regimen for treating a disease condition with the compounds and/or compositions of this invention is selected in accordance with a variety of factors, including the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized and whether the compound is administered as part of a drug combination. Thus, the dosage regimen actually employed may vary widely and therefore may deviate from the preferred dosage regimen set forth above.

The compounds of the present invention may be administered orally, parenterally, by inhalation spray, rectally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous injections, intravenous, intramuscular, intrasternal injection, or infusion techniques.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols which are solid at ordinary temperatures but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally by prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

While the compounds of the invention can be administered as the sole active pharmaceutical agent, they can also be used in combination with one or more therapeutic agents, such as immunomodulators, antiviral agents or antiinfective agents.

The foregoing is merely illustrative of the invention and is not intended to limit the invention to the disclosed compounds. Variations and changes which are obvious to one skilled in the art are intended to be within the scope and nature of the invention which are defined in the appended claims. From the foregoing description, one skilled in the art can easily ascertain the essential characteristics of this invention, and without departing from the spirit and scope thereof, can make various changes and modifications of the invention to adapt it to various usages and conditions.

All documents referred to herein are specifically incorporated herein by reference in their entireties, including the priority document, U.S. Provisional Application No. 60/352,881, filed Feb. 1, 2002, which is incorporated herein by reference in its entirety.

1. A compound represented by the following structure:
R¹ —X—R²
wherein,
X is a hydrophilic polymer selected from the group consisting of polyoxazoline, polyethylene glycol, polyacetal, polylactic acid, and polyglycolic acid;
R¹ is a hydroxyl group, a sulfhydryl group, carboxylic acid, carboxylic acid ester, amino, amide group, a cell targeting ligand, or a tissue targeting ligand; and
R² may be a therapeutic agent selected from the group consisting of a peptide, polypeptide, protein, nucleic acid and a therapeutic drug,

2. A compound represented by the following structure:
R¹—X—R²
wherein,
X is a hydrophilic polymer selected from the group consisting of polyoxazoline, polyethylene glycol, polyacetal, polylactic acid, polyglycolic acid,
R1 is a therapeutic agent selected from the group consisting of a peptide, polypeptide, protein, nucleic acid and a therapeutic drug; and
R2 is a hydroxyl group, a sulfhydryl group, carboxylic acid, carboxylic acid ester, amino, amide group, a cell targeting ligand, or a tissue targeting ligand.

3. A compound represented by the following structure: 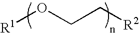
wherein,
R¹ is a therapeutic agent selected from the group consisting of a peptide, polypeptide, protein, nucleic acid and a therapeutic drug;
R² is a hydroxyl group, a sulfhydryl group, carboxylic acid, carboxylic acid ester, amino, amide group, a targeting moiety, a fusogenic moiety or a nuclear targeting moiety, and
n=1-500.

4. A compound represented by the following structure: 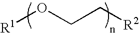
wherein,
R¹ is a hydroxyl group, a sulfhydryl group, carboxylic acid, carboxylic acid ester, amino, amide group, a cell targeting ligand, or a tissue targeting ligand;
R² is a therapeutic agent selected from the group consisting of a peptide, polypeptide, protein, nucleic acid and a therapeutic drug, and
n=1-500.

5. A compound represented by the following structure: 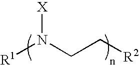
wherein,
X is —CO—R, —(CH₂)ₘ—COOH, wherein m is an integer 1-25, (CH₂)ₚ—OH, wherein p is an integer 1-25, —(CH₂)_{q}—COOH, wherein q is an integer 1-25, an ester group, polyethylene glycol, polylactic acid, polyglycolic acid, polyoxazoline, amino, imidazole, or guanidinium; wherein R may be —CH₃, —C₂H₅, —(CH₂)ᵣ—OH, wherein r is an integer 1-25;
R¹ is a hydroxyl group, a sulfhydryl group, carboxylic acid, carboxylic acid ester, amino, amide group, a targeting moiety, a fusogenic moiety, or a nuclear targeting moiety; and
R² is a therapeutic agent selected from the group consisting of a peptide, polypeptide, protein, nucleic acid or a therapeutic drug, and
n is 1-500.

6. A compound represented by the following structure: 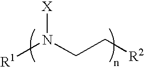
wherein,
X is —CO—R, —(CH₂)ₘ—COOH, wherein m is an integer 1-25, (CH₂)ₚ—OH, wherein p is an integer 1-25, —(CH₂)_{q}—COOH, wherein q is an integer 1-25, an ester group, polyethylene glycol, polylactic acid, polyglycolic acid, polyoxazoline, amino, imidazole, or guanidinium; wherein R may be —CH₃, —C₂H₅, —(CH₂)ᵣ—OH, wherein r is an integer 1-25;
R¹ is a therapeutic agent selected from the group consisting of a peptide, polypeptide, protein, nucleic acid or a therapeutic drug; and
R² is a hydroxyl group, a sulfhydryl group, carboxylic acid, carboxylic acid ester, amino, an amide group, a targeting moiety, a fusogenic moiety, or a nuclear targeting moiety; and
n=1-500.

7. A compound represented by the following structure: 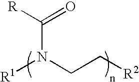
wherein,
R is —CH₃, —C₂H₅, a hydrocarbon with 1-18 carbons, (C₁-C₂₅)—OH, (C₁-C₂₅)—COOH, polyethyleneglycol, polylactic acid, polyglycolic acid, polyoxazoline;
R¹ is a hydroxy group, a sulfhydryl group, carboxylic acid, carboxylic acid ester, amino, amide group, a cell targeting ligand, or a tissue targeting ligand; and
R² is a therapeutic agent selected from the group consisting of a peptide, polypeptide, protein, nucleic acid or a therapeutic drug, and
n=1-500.

8. A compound represented by the following structure: 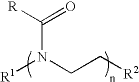
wherein,
R is —CH₃, —C2H₅, a hydrocarbon with 1-18 carbons, (C₁-C₂₅)—OH, (C₁-C₂₅)—COOH, polyethyleneglycol, polylactic acid, polyglycolic acid, polyoxazoline;
R¹ is a therapeutic agent selected from the group consisting of a peptide, polypeptide, protein, nucleic acid or a therapeutic drug;
R² is a hydroxyl group, a sulfhydryl group, carboxylic acid, carboxylic acid ester, amino, amide group, a targeting moiety, a fusogenic moiety or a nuclear targeting moiety, and
n=1-500.

9. A method of treating a subject suffering from a disorder treatable by a therapeutic agent, comprising administering to said subject an effective amount of a compound according to any one of claims 1-8.

10. A method delivering a therapeutic agent to a subject in need thereof comprising administering to a subject in need thereof an effective amount of a compound according to any one of claims 1-8.
